(19) United States
(12) Patent Application Publication
(10) Pub. No.: US 2014/0257036 A1
(43) Pub. Date: Sep. 11, 2014

(54) Method And Device For Detecting Thermal Effects Of Tissue Ablation On A Tissue Of A Patient

(71) Applicant: Ohio University, Athens, OH (US)

(72) Inventors: JungHun Choi, Athens, OH (US); Randolph B. Robert, III, Athens, OH (US)

(73) Assignee: Ohio University, Athens, OH (US)

(21) Appl. No.: 14/203,990

(22) Filed: Mar. 11, 2014

Related U.S. Application Data
(60) Provisional application No. 61/776,376, filed on Mar. 11, 2013.

(51) Int. Cl. A61B 18/12

(52) U.S. Cl. CPC A61B 18/1233
USPC 600/104; 606/34

(57) ABSTRACT

A method and device for detecting thermal effects of tissue ablation on a tissue of a patient includes an endoscope, a biopsy forceps, and an electrical measurement circuit. The method includes measuring a first value of an electrical property of the tissue and removing at least a portion of the tissue with the hot biopsy forceps. The method also includes measuring a second value of the electrical property of the remaining tissue. Accordingly, the electrical measurement circuit is configured to correlate the difference between the first and second values to a depth of ablation.

### CROSS-REFERENCE TO RELATED APPLICATION

This application claims the priority of application Ser. No. 61/776,376 filed Mar. 11, 2013 (pending), the disclosure of which is hereby incorporated by reference herein.

### TECHNICAL FIELD

The present invention relates generally to a method and device for detecting thermal effects of tissue ablation on a tissue of a patient, and more particularly, to a method and device for detecting the thermal effects of tissue ablation to inhibit tissue damage of the patient during a colonoscopy.

### BACKGROUND

One of the most common and dangerous electrosurgical procedures is monopolar electrosurgery for removing polyps, such as colorectal polyps, with an endoscope and hot biopsy forceps. While such biopsy forceps are widely used for removing polyps that may be associated with colorectal cancer, successfully removing polyps with minimal damage to a patient's tissue requires significant training to properly inspect an ablation region for thermal damage. For example, a practitioner, such as a doctor, nurse, or other trained medical professional, typically visually inspects the ablation region for growth of a white peripheral crest to indicate a depth of the thermal effects into the tissue caused by the ablation of the polyp.

On the one hand, visually overestimating the depth of the thermal effects of the ablation may lead the practitioner to incorrectly conclude that the polyp has been completely removed by the biopsy forceps and, in turn, may fail to fully remove the polyp. On the other hand, visually underestimating the depth of the thermal effects of the ablation may cause the practitioner to inadvertently damage the patient's tissue resulting in potentially life-threatening complications. Such underestimations are further complicated by the fact that these complications may include a delayed perforation of the patient's tissue after the patient as left the practitioner and is no longer surrounded by trained medical professionals for immediate treatment.

There is a need for a method of detecting the thermal effects of tissue ablation on a tissue of the patient, such as removing a polyp during a colonoscopy, that addresses present challenges and characteristics such as those discussed above.

### SUMMARY

An exemplary embodiment of a method for detecting thermal effects of tissue ablation on a tissue of a patient includes measuring a first value of an electrical property of the tissue before removing at least a portion of the tissue with a hot biopsy forceps at an ablation region. The method also includes removing at least the portion of the tissue with the hot biopsy forceps at the ablation region having a remaining portion of the tissue. In addition, the method includes measuring a second value of the electrical property of the remaining tissue and correlating the difference between the first and second values to a tissue damage depth.

An exemplary embodiment of a device for detecting thermal effects of tissue ablation on a tissue of a patient during a colonoscopy includes an endoscope, a biopsy forceps, and an electrical measurement circuit. The endoscope has a shaft extending to an end portion. The endoscope is also operatively connected to an endoscope control configured for operating the endoscope during the colonoscopy. The biopsy forceps has a head configured for removing at least a portion of the tissue at an ablation region. The biopsy forceps extend through the end portion of the endoscope and are operatively connected to a biopsy forceps control for operating the biopsy forceps during the colonoscopy. The electrical measurement circuit is operatively connected to the biopsy forceps and configured to detect an electrical property of the ablation region.

Various additional objectives, advantages, and features of the invention will be appreciated from a review of the following detailed description of the illustrative embodiments taken in conjunction with the accompanying drawings.

### BRIEF DESCRIPTION OF THE DRAWINGS

The accompanying drawings, which are incorporated in and constitute a part of this specification, illustrate embodiments of the invention and, together with a general description of the invention given above, and the detailed description given below serve to explain the invention.

FIG. 1 is a schematic view of an embodiment of an endoscope having a biopsy forceps configured for detecting effects of tissue ablation on a tissue of a patient.

FIG. 2 is diagrammatic view of an embodiment of a test assembly configured to detect effects of tissue ablation on a test tissue.

FIG. 3 is a chart showing capacitance measurements collected at the test tissue during operation of the test assembly of FIG. 2.

FIG. 4 is a chart showing an ablation diameter collected at the test tissue during operation of the test assembly of FIG. 2.

### DETAILED DESCRIPTION

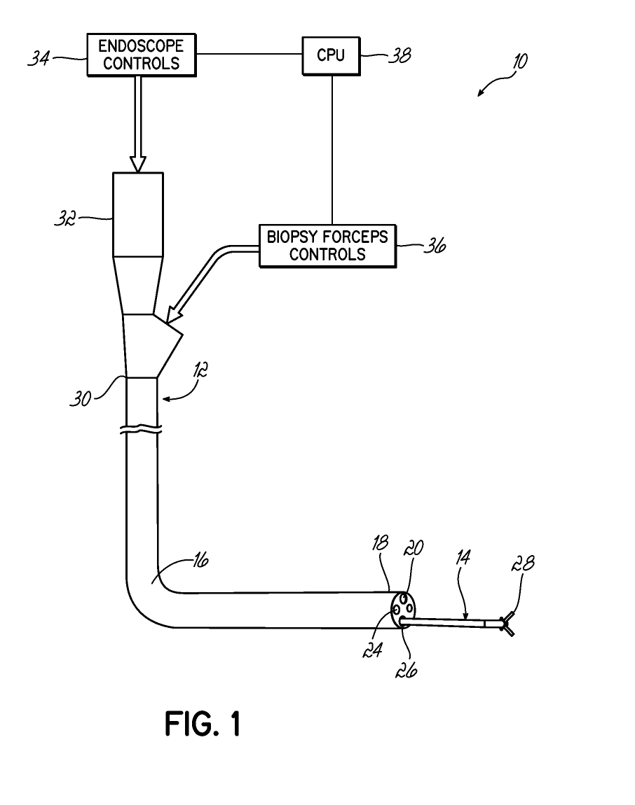

With reference to FIG. 1, an embodiment of a device 10 for performing an electrosurgical procedure, particularly with respect to identifying and removing a polyp tissue from a colon tissue of a patient during a colonoscopy, includes an endoscope 12 and a hot biopsy forceps 14 configured for removing the polyp tissue. As described herein, a portion of patient tissue identified for removal of the polyp tissue is referred to as an “ablation region” of tissue and may include the polyp tissue and/or any remaining tissue after removal of the polyp tissue. The endoscope 12 includes a flexible shaft 16 having a distal end portion 18. The distal end portion 18 includes a camera 20, or similar visualization equipment, an illumination device 24, such as a light bulb, and a channel 26 through which the hot biopsy forceps 14 extends to access the polyp tissue. A head 28 of the hot biopsy forceps 26 is configured to detect an electrical property of the ablation region for determining the effects of ablation on the tissue. The effects of ablation refer to a wound cavity formed within an ablation region by the removal of the polyp. The wound cavity has a depth and a diameter, and may vary in size depending on the amount of tissue removed during the ablation. Generally, a wound cavity having a relatively large depth is more severe with an increased risk of perforating the tissue than a wound cavity having a relative shallow depth. By correlating the electrical property values to the depth of the wound cavity, a practitioner, such as a doctor, nurse, or other medical professional, may more fully understand the effects of ablation in real-time for removing the polyp tissue while also reducing the likelihood of overestimating or underestimating the depth of ablation at the ablation region.

The endoscope 12 further includes a proximal end portion 30 having a handle 32 by which the practitioner may manipulate the endoscope 12 to identify the polyp tissue during the colonoscopy. The proximal end portion 30 is also operatively connected to endoscope controls 34 configured for operating the endoscope and biopsy forceps controls 36 configured for operating the biopsy forceps 36. While the connection of the endoscope and biopsy controls 34, 36 to the endoscope 12 and the biopsy forceps 14 will not be described in detail herein, it will be appreciated that the general operation of the endoscope 12 and biopsy forceps 14 are well known in the medical field.

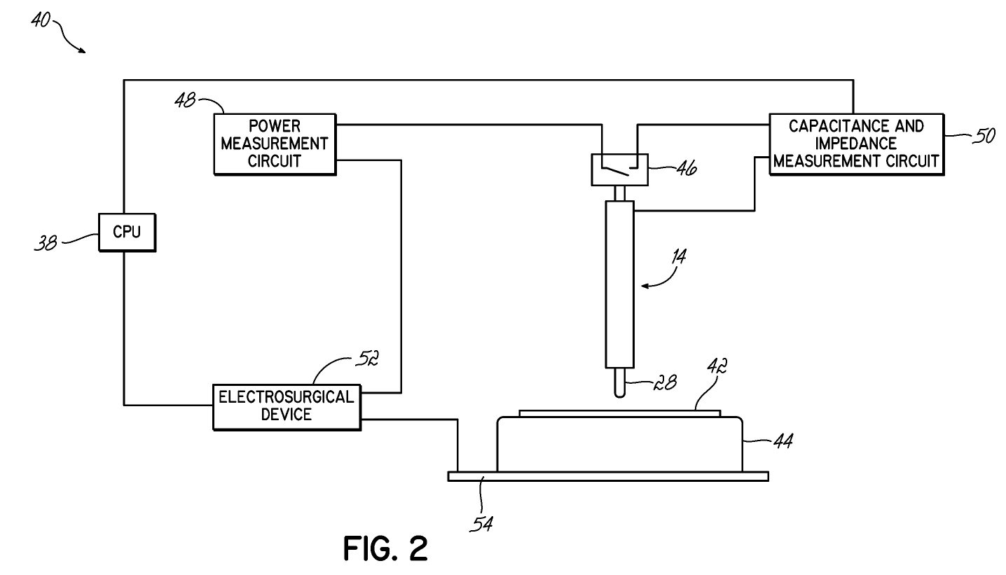

As shown in FIGS. 1 and 2, a CPU 38 is operatively connected to various components for operating the endoscope 12 and the biopsy forceps 14, collecting detected measurements, and correlating detected measurements to the depth of ablation. More particularly, FIG. 2 represents the endoscope 12 and the biopsy forceps 14 in the form of a test assembly 40 configured to detect the depth of tissue ablation on an ex vivo test tissue 42. The following will address these effects with respect to the test assembly 40 rather than the endoscope 12 of FIG. 1. However, it will be appreciated that all of the test assembly 40 or one or more portions of the test assembly 40 may be integrated into the endoscope and/or biopsy forceps controls 34, 36 and connected to the CPU 38 for similarly determining the depth of tissue ablation on the patient, such as during the colonoscopy.

With respect to FIG. 2, the patient is simulated by the test tissue 42, such as a portion of a pig colon, and a gel block 44 configured to replicate the electrical properties of the patient during the electrosurgical procedure. The test assembly 40 replicates the biopsy forceps 14 and head 28, which contacts the test tissue 42 to thermally remove a portion of the test tissue 42 by ablation. The test assembly 40 also includes a switch 46 for selectively powering the biopsy forceps 14, a power measurement circuit 48, an electrical measurement circuit 50, and an electrosurgical device 52. According to an exemplary embodiment, the electrical measurement circuit 50 is in the form of a capacitance and impedance measurement circuit 50 for measuring the electrical properties of capacitance and impedance. However, another electrical property, such as resistance or voltage, may be used for detecting the depth of tissue ablation as described below with respect to capacitance and impedance. While the exemplary embodiment of the electrical measurement circuit 50 measures both capacitance and impedance, it will be appreciated that the electrical measurement circuit 50 may alternatively measure only one electrical measurement, such as capacitance and impedance.

As discussed briefly above, the depth of tissue ablation correlates to an electrical property of the ablation region during the electrosurgical procedure. To replicate the electrosurgical procedure with the test assembly 40, the electrosurgical device 52 directs a desirable operating power to the power measurement circuit 48, through the connected switch 46, and electrically powers the head 28 of the biopsy forceps 14. While being electrically powered, the head 28 is brought into contact with the test tissue 42 to perform the ablation. The capacitance and impedance measurement circuit 50 is operatively connected to a base 54 electrically connected to the gel block 44 opposite the test tissue 42 for detecting the capacitance and impedance across the entirety of the ablation region. As such, the capacitance and impedance measurements may be collected before, after, or during the ablation of the test tissue 42.

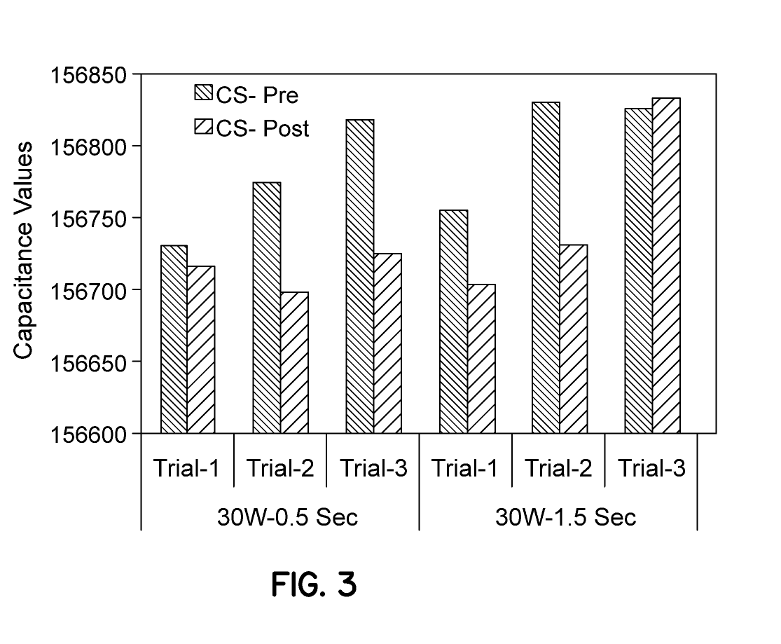

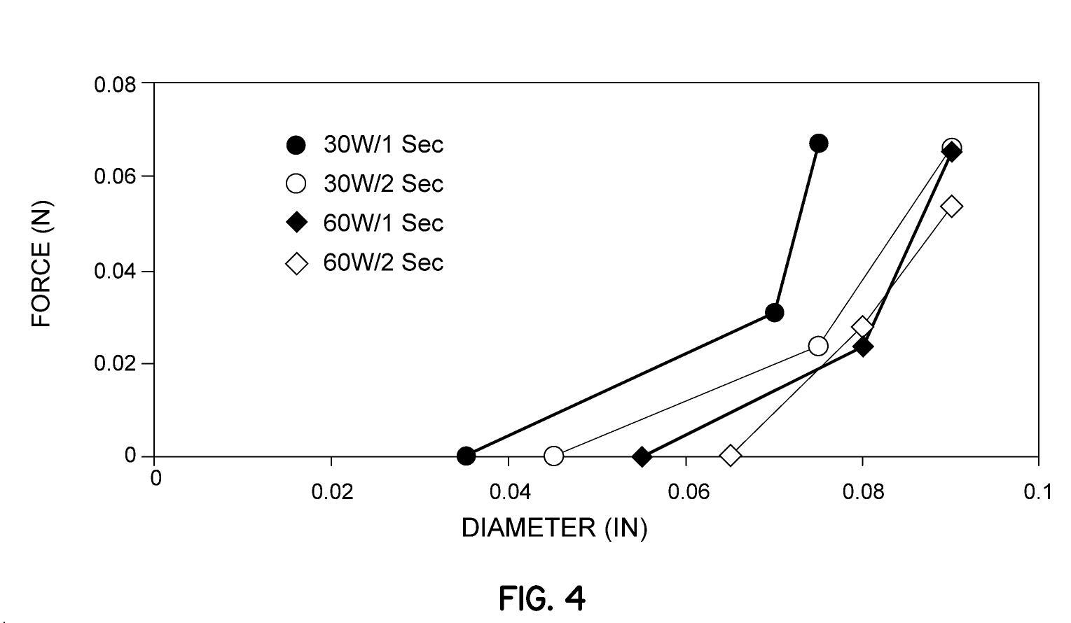

According to an exemplary embodiment, the electrosurgical device 52, the power measurement circuit 48, the capacitance and impedance measurement circuit 50, and the base 54 are operatively connected to the CPU 38 for measuring values of the detected capacitance and the detected impedance. The CPU is further configured to correlate the measured values of capacitance and impedance to the depth of ablation. In this respect, FIGS. 3-4 show various test results of the test assembly 40 indicative of the electrical property values and diameter of the ablation on the test tissue 42.

With respect to FIG. 3, the electrical property is capacitance and indicates damage to the test tissue occurring in real-time for both 0.5 second and 1.5 seconds. The capacitance values are unitless, and relate to the impedance and the amount of time required to charge the test tissue 42. Furthermore, FIG. 4 shows a variation in diameter of the ablation taken at 1 second and 2 second intervals for both 30 watts and 60 watts of power. The following tables further detail these results as a comparison to the various dimensions of the ablation that occurred during these various trials.

TABLE 1
Histology Analysis of Tissue Damage at 0.5 seconds

| Trial | Diameter (mm) | Depth (mm) |
|---|---|---|
| 1 | 0.382 | 0.085 |
| 2 | 0.403 | 0.090 |
| 3 | 0.410 | 0.091 |

TABLE 2
Histology Analysis of Tissue Damage at 1.5 seconds

| Trial | Diameter (mm) | Depth (mm) |
|---|---|---|
| 1 | 0.494 | 0.109 |
| 2 | 0.489 | 0.110 |
| 3 | 0.442 | 0.113 |

With respect to FIGS. 1-4, Table 1, and Table 2, the diameters and depth of ablation correlate to the capacitance and impedance values for each trial. While the above ablation data collected with the test assembly 40 represents only a small sample size, it will be appreciated that testing may be repeated and the sample size increased for improved correlation between these measurements and the depth of the wound cavity from ablation of a polyp tissue.

In any case, the CPU 38 collects such measurements and correlates these measurements for aiding the practitioner in understanding the depth of ablation on the patient. Thereby, the CPU 38 determines the amount of thermal energy delivered into the ablation region. According to an exemplary embodiment, the measurements of capacitance and impedance are taken before and after each ablation to be compared by the CPU 38 for correlating the difference between the values and indicating the depth of the tissue before and after each ablation.

While the present invention has been illustrated by the description of one or more embodiments thereof, and while the embodiments have been described in considerable detail, they are not intended to restrict or in any way limit the scope of the appended claims to such detail. For example, the references to the colonoscopy procedure and the polyp tissue are not intended to limit the invention. It will be appreciated that the invention may be used in any electrosurgical procedure and on any patient tissue. The various features shown and described herein may be used alone or in any combination. Additional advantages and modifications will readily appear to those skilled in the art. The invention in its broader aspects is therefore not limited to the specific details, representative device and method and illustrative examples shown and described. Accordingly, departures may be from such details without departing from the scope of the general inventive concept.

What is claimed is:

1. A method of detecting thermal effects of tissue ablation on a tissue of a patient, comprising;
measuring a first value of an electrical property of the tissue before removing at least a portion of the tissue with a hot biopsy forceps at an ablation region;
removing at least the portion of the tissue with the hot biopsy forceps at the ablation region having a remaining portion of the tissue;
measuring a second value of the electrical property of the remaining tissue; and
correlating the difference between the first and second values to a depth of ablation.

2. The method of claim 1 wherein the electrical property of the tissue is capacitance.

3. The method of claim 1 further comprising repeating measuring the first value, removing at least the portion of the tissue, measuring the second value, and correlating the difference between the first and second values to the depth of ablation.

4. The method of claim 1 wherein the portion of the tissue is a polyp tissue and further comprises:
performing a colonoscopy on the patient with an endoscope having a visualization equipment and the hot biopsy forceps; and
identifying the polyp tissue within a colon of the patient with the visualization equipment.

5. A device for detecting thermal effects of tissue ablation on a tissue of a patient during a colonoscopy, comprising;
an endoscope having a shaft extending to an end portion, the endoscope operatively connected to an endoscope control configured for operating the endoscope during the colonoscopy;
a biopsy forceps having a head configured for removing at least a portion of the tissue at an ablation region, the biopsy forceps extending through the end portion of the endoscope and operatively connected to a biopsy forceps control for operating the biopsy forceps during the colonoscopy; and
an electrical measurement circuit operatively connected to the biopsy forceps and configured to detect an electrical property of the ablation region.

6. The device of claim 5 wherein the endoscope further includes visualization equipment for identifying the tissue for tissue ablation.

7. The device of claim 5 wherein the electrical measurement circuit is a capacitive measurement circuit configured to measure a capacitive value of the ablation region.

8. The device of claim 7 further comprising:
a CPU operatively connected to the capacitive measurement circuit and configured to correlate the measured capacitance value to a depth of ablation for detecting the effects of ablation on the patient.
